(19) United States
(12) Patent Application Publication
(10) Pub. No.: US 2014/0336724 A1
(43) Pub. Date: Nov. 13, 2014

(54) RETINAL PROSTHESIS

(71) Applicants: NATIONAL ICT AUSTRALIA LIMITED, Eveleigh (AU); CENTRE FOR EYE RESEARCH AUSTRALIA LIMITED, East Melbourne (AU); THE BIONICS INSTITUTE OF AUSTRALIA, East Melbourne (AU)

(72) Inventors: David Chee Keong Ng, Pascoe Vale South (AU); Shun Bai, Parkville (AU); Efstratios Skafidas, Thornbury (AU); Penelope Allen, Kew (AU); Chris Williams, East Melbourne (AU); Hamish Meffin, Braybrook (AU); Mark McCombe, Kew (AU); Mark Edward Halpern, East Malvern (AU); Clive Steward Boyd, Mooroolbark (AU)

(21) Appl. No.: 14/288,147

(22) Filed: May 27, 2014

Related U.S. Application Data
(62) Division of application No. 13/266,317, filed on Jan. 9, 2012.

(30) Foreign Application Priority Data
Apr. 27, 2009 (AU) 2009901812

(51) Int. Cl. A61N 1/36; A61N 1/372; A61N 1/378

(52) U.S. Cl. CPC A61N 1/36046; A61N 1/3787; A61N 1/37229
USPC 607/54

(57) ABSTRACT

The present invention relates to retinal prostheses, and in particular to the transfer of electrical power and data from outside of the human body to such a prosthesis. The retinal prosthesis comprises: A retinal electrode array implanted in the eye to stimulate the retina. A receiving coil implanted sub-sclerally to inductively receive power or data signals, or both. An electrical connection between the implanted receiving coil and the implanted retinal electrode array. Wherein the receiving coil is flexible and able to conform to scleral curvature, when it is implanted. And wherein power or data signals, or both, received by the receiving coil from a remote transmitting coil are automatically provided to the electrode array. According to a second aspect, the present invention provides a method for implanting a retinal prosthesis. In a further aspect the present invention further provides an ocular implant.

### CROSS REFERENCE TO RELATED APPLICATIONS

This application is a division of application Ser. No. 13/266,317 filed Oct. 26, 2011 for Retinal Prostheses which is a national application of PCT application number PCT/AU2010/000474 filed Apr. 23, 2010 for Retinal Prosthesis, which claims priority from Australian application number 2009/901812 filed Apr. 27, 2009 for Retinal Prosthesis.

### BACKGROUND OF THE INVENTION

1. Field of the Invention

The present invention relates to retinal prostheses, and in particular it relates to the transfer of electrical power and data from outside of the human body to such a prosthesis.

2. Description of Related Art

A retinal prosthesis which uses electrical signals to stimulate the retina requires electrical power to function. The power demands of retinal prostheses are likely to increase as higher density electrode arrays are developed in future. A retinal prosthesis cannot practically be powered by wires from an external power supply, as any wired connection through the skin creates a risk of infection and causes discomfort to the user. Previous proposals for powering retinal prostheses include the use of optical and acoustic energy, however these solutions are ineffective due to high losses when these energy types pass through tissue.

Inductive coupling links have also been proposed for visual prostheses. Such proposals provide for an external primary coil which is placed in front of the eye, and a secondary internal coil inside the eye. However, the large separation between these coils and the small diameter of the internal coil result in low power transfer efficiency.

Surgical implantation of a prosthesis into the eye also presents particular difficulties due to the need to ensure ongoing integrity of the ocular structure, at least after surgical healing is complete. For example the sclera provides resistance to forces on the eye, helps to maintain intraocular pressure, and helps resist infection within the eye. Any implanted device must also be structured and positioned in a way to allow for the normal range of motion of the eyeball and the almost continual nature of eyeball movement.

### SUMMARY OF THE INVENTION

According to a first aspect the present invention provides a retinal prosthesis, comprising:

A retinal electrode array implanted in the eye to stimulate the retina.

A receiving coil implanted sub-sclerally to inductively receive power or data signals, or both.

An electrical connection between the implanted receiving coil and the implanted retinal electrode.

Wherein the receiving coil is flexible and able to conform to scleral curvature, when it is implanted, and wherein power or data signals, or both, received by the receiving coil from a remote transmitting coil are automatically provided to the electrode array.

The sub-scleral positioning of the receiving coil enables a retinal prosthesis to be implemented which has no wiring or tethers from the intraocular devices to the extraocular space. This allows for complete healing of the sclera after the device is implanted, which allows the sclera to perform its role providing a barrier to intraocular infection.

The retinal electrode array may be sub-retinal, for instance it may be located epi-retinally near the fovea in the macular region of the retina.

One or more retinal self-locking tacks may be used to mechanically secure the electrode array in the epi-retinal position. A retinal tack may also complete an electrical connection between the secondary coil and the electrode array. The retinal tack may be formed of an insulating material, such as a ceramic, and house a conductor that spans electrical contacts at each end of the tack.

In one example the retinal tack may be arranged to conduct power or data signals, or both, from the secondary coil through the choroid or retina, or both, to the electrode array. The insulating material of the retinal tack may minimize electrical current leakage between the secondary coil and the electrode array. The conductor of the retinal tack may carry data signals in both directions, to and from the retinal array.

Other locations for the retinal electrode array are also possible, for instance in the suprachoroidal space.

The receiving coil (generally nominated the ‘secondary’ coil below) may be intrinsically flexible. Alternatively, it may be formed or mounted on a flexible substrate.

The receiving coil may be located between the sclera and the choroid. It may be located supra-choroidally and epi-sclerally.

The present invention thus provides for an implanted secondary coil which does not require a full sclerotomy, and as a result does not result in an interconnect piercing the sclera.

The receiving coil may be connected to the electrode array by the use of flexible wiring. The flexible wiring may be implanted sub-sclerally (in the suprachoroidal layer) between the coil and the array (or a retinal tack). Alternatively, the wiring from the receiving coil may intrude through the choroid and the vitreous humor to the array. In this case it is preferred that the wiring does not pass through any part of the retina.

The receiving coil may receive power and data signals by inductive coupling with a transmitting coil.

The remote transmitting (primary) coil may be located externally to the body, for instance mounted on the surface of the skin.

In some situations it may be necessary or desirable to use one or more additional, intermediate coils to relay power or data signals, or both, from the remote transmitting (primary) coil to the receiving (secondary) coil. For instance a first intermediate coil may be implanted in the zygomatic bone for receiving power from an external primary coil. A second intermediate coil may be implanted in the orbit of the ocular region for inductively relaying power from the first intermediate coil to the secondary. The first and second intermediate coils may be in wired connection, which improves transmission efficiency.

According to a second aspect, the present invention provides a method for implanting a retinal prosthesis, comprising:
Implanting a retinal electrode array in the eye.
Implanting a flexible receiving coil sub-sclerally, wherein the receiving coil is flexible and able to conform to scleral curvature when it is implanted.

Electrically connecting the retinal electrode array to the implanted receiving coil, such that power or data signals, or both, received by the receiving coil from a remote transmitting coil are automatically provided to the electrode array.

In a further aspect the present invention further provides an ocular implant, comprising:

A receiving coil for implanting sub-sclerally to inductively receive power or data signals, or both.

A retinal electrode array.

Flexible wiring interconnecting the receiving coil and the electrode array.

Wherein the receiving coil and the wiring are flexible and at least the receiving coil is able to conform to scleral curvature, when implanted.

### BRIEF DESCRIPTION OF THE DRAWINGS

The exact nature of this invention, as well as the objects and advantages thereof, will become readily apparent from consideration of the following specification in conjunction with the accompanying drawings in which like reference numerals designate like parts throughout the figures thereof and wherein:

FIGS. 1(a) and 1(b) are diagrams that illustrate the anatomical positioning of a secondary coil for effecting an inductive power link to a retinal implant;

FIG. 2(a) is a diagram that illustrates the location of an external primary coil;

FIG. 2(b) is a diagram that illustrates the relative locations of the external primary coil of FIG. 2(a), two internal intermediate coils, and the internal secondary coil of FIGS. 1;

FIG. 3(a) is cross-section of an eye from the side, showing the location of a retinal electrode array and the relative locations of the external primary coil and the internal secondary coil that are in front of the plane of the section, and the route of the flexible wiring interconnecting the internal secondary coil and the retinal electrode array;

FIG. 3(b) is a magnified section through the layers of the eyeball showing the locations of the internal secondary coil and the flexible wiring connected to it;

FIG. 3(c) is a magnified section through the layers of the eyeball of FIG. 3(b) showing the locations of the retinal electrode array, a retinal tack and the flexible wiring from the internal secondary coil;

FIG. 4(a) is cross-section of an eye from the side, showing the location of a retinal electrode array and the relative locations of the external primary coil and the internal secondary coil that are in front of the plane of the section, and an alternative route of the wiring interconnecting the internal secondary coil and the retinal electrode array;

FIG. 4(b) is a magnified section through the layers of the eyeball of FIG. 4(a) showing the locations of the internal secondary coil and the wiring connected to it;

FIGS. 5(a) and (b) show a variation of the arrangement of FIG. 4 where the internal secondary coil is mounted on a flexible scleral buckle that is positioned episclerally;

FIG. 6 is a diagram of the eyeball showing the location of the surgical incision required for a trans-vitreous connection to be made between the internal secondary coil and the retinal electrode array;

FIG. 7 is a variation on FIG. 2(b) where there is only a single intermediate coil;

FIG. 8(a) is a diagram of a first configuration for a power transfer coil;

FIG. 8(b) is a diagram of a second configuration for a power transfer coil; and

FIG. 8(c) is a diagram of a third configuration for a power transfer coil.

### DETAILED DESCRIPTION OF THE PREFERRED EMBODIMENTS

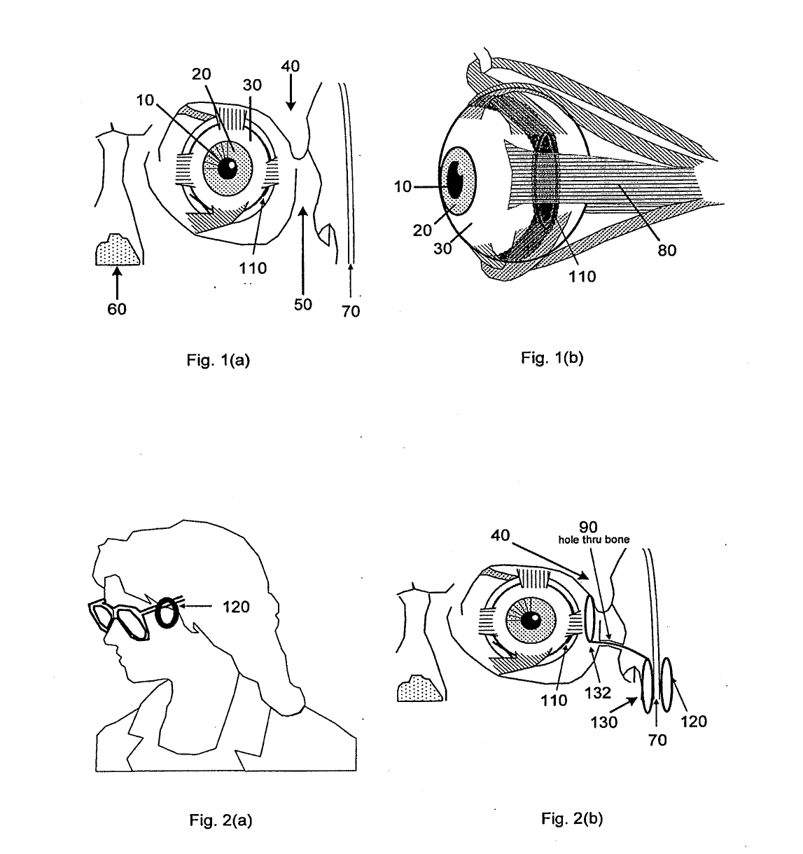

Referring first to FIGS. 1(a) and 1(b), these diagrams illustrate the anatomical location of a secondary coil 110 for effecting an inductive power link to a retinal implant (not shown). FIG. 1(a) shows the eye ball from the front, and FIG. 1(b) from the side, with the pupil 10, iris, 20 and the sclera (white of the eye) 30. The bony structure around the eye is also indicated at 40 in FIG. 1(a), as is the zygomatic bone 50, the nose 60 and the skin on the temple 70. Several muscles that move the eye (such as the ciliary muscle 80) are also shown in both drawings but they are not important for understanding the location of the secondary coil 110. The secondary coil is shown at the side of the eye but is implanted beneath the sclera 30, and would not be visible to an observer.

FIG. 2(a) illustrates the location of an external primary coil 120, close to the temple. The primary coil 120 is located externally on or near the skin 70, and is connected to a power source (not shown). The secondary coil 110 is connected to a retinal implant (not shown) inside the eye and is located on a flexible substrate, that is implanted at a location under the sclera.

An inductive link operates by a time-varying electrical current flowing in a primary coil inducing a current in a secondary coil, provided it is located in sufficiently close proximity to the primary coil. In this case, the primary coil 120 is located outside the eye, while the secondary coil 110 is placed inside the eye. The primary coil 120 is connected to a power source via an electrical connection and electronics that drive the current in the coil. The secondary coil 110 is connected to an implanted device via electronics which recover the energy from the inductive link to supply power to the implant. The inductive link removes the need for a physical (wired) electrical connection between an external power source and the implanted device.

An inductive power link for a retinal prosthesis, must take into account the fact that the secondary coil 110 is constrained by the physical and biological environment of the eye, and the high power consumption of the implanted device. The aim is to deliver the required power without exceeding safety limits.

The secondary coil 110 is formed or mounted upon a flexible substrate which is surgically implanted in the supra-choroidal space. In particular the secondary coil 110 is provided on a thin flexible circuit, or substrate, much like a scleral buckle. This entire device is surgically implanted under the sclera, between the sclera and the choroid in the suprachoroidal space. Furthermore, the secondary coil is located near the ciliary muscle 80 (see FIG. 1(b)), away from the retina, to prevent injury to the delicate retina 400 layer.

FIG. 2(b) illustrates the relative locations of the external primary coil 120, two internal intermediate coils 130 and 132, as well as the internal secondary coil 110. The intermediate coils 130 and 132 are used to improve the power transfer efficiency between the primary coil 120 and the secondary coil 110. The first intermediate coil 130 sits on the zygomatic bone 50 of the skull immediately adjacent the external primary 120. The second intermediate coil 132 sits in the orbit of the ocular region immediately adjacent the secondary 110. These two coils are in circuit with each other via a hole 90 through the zygomtic bone.

Due to the relatively small distance between the secondary coil 110 and the second intermediate coil 132, and the relatively small distance between the primary coil 120 and the first intermediate coil 130, more efficient power transfer can be achieved. Moreover, such efficiency is achieved in a manner which provides wireless power transfer through the sclera 30, allowing the sclera to be maintained intact once healed from implantation surgery. Further, the relative positioning of second intermediate coil 132 and secondary 110 allows for normal movement of the eyeball, while maintaining high power transfer efficiency which is relatively unsusceptible to movements of the eye.

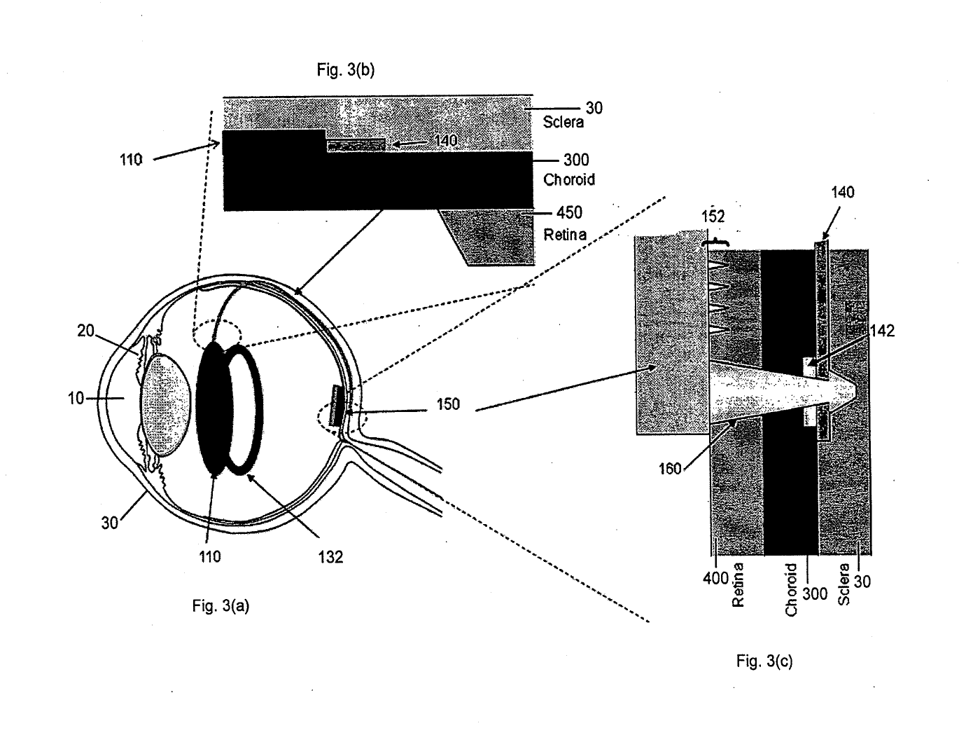

The retinal electrode array 150 is implanted epi-retinally near the fovea in the macular region of the retina. The retinal array is held in place by a self-locking retinal tack 160, seen in FIG. 3(c). Also shown in FIG. 3(c) are a number of retina stimulating electrodes 152 extending from the back of the electrode array 150 into the tissue of the retina.

Power is transferred from the secondary coil 110 to the retinal electrode array 150 by a physical connection comprising flexible circuit wiring 140. In FIGS. 3(a) and (b) the flexible circuit wiring 140 is disposed along the suprachoroidal space between the secondary coil 110 and the retina, where is passes behind the retinal electrode array 150. To connect the power from the wiring 140 in the suprachoroidal space through the choroid 300 and retina 400 to the implant 150, it passes through a conductive path in the retinal tack 160.

The use of a retinal tack to both mechanically anchor the implant 150, and also provide an electrical connection from the wiring 140 to the implant, minimizes the number of components piercing the retina 400 and choroid 300.

It is envisaged that more than one tack may be required to adequately anchor implant 150. In the case where more than one tack 160 is used, some or all of the tacks may provide electrical connections to the wiring 140.

In order to prevent current leakage from the conductive path of the retinal tack 160, it is sheathed in non-conductive ceramic from the contact point 142 between the tack 160 and the flexible wiring 140. The conductor (not shown) of the retinal tack 160 then efficiently conveys power, and if required a data signal, from the electrical wiring 140 to the retinal implant 150 with minimal leakage. The power and data received by the electrode array 150 enables electrical stimuli to be applied via electrodes 152 to the nerve cells in the retina.

In order to locate the inductive coils 110, 120, 130 and 132 in and near the eye, the following surgical procedure is followed:

First, the flexible wiring 140 is inserted into the suprachoroidal space near the macular region of the eye.

Next, the secondary coil 110 is placed under the sclera 30.

Once the sclera 30 has recovered reasonably well, the retinal implant unit 150 is inserted by performing an incision at the pars plana region and securing it in place with the self-locking tacks 160.

Next, the intermediate coils 130 and 132 are anchored to the zygomatic bone with one coil in the ocular region and the other coil under the skin.

The intermediate coils 130 and 132 are connected via a physical wire which passes through a hole bored through the zygomatic bone, as shown in FIG. 2(b).

A wireless power link is then established from the intermediate coil 130 to the primary coil 120, outside the body. The primary coil could be carried by, or encased within, the frame of a pair of spectacles, as shown in FIG. 2(a).

The sub-scleral positioning of the secondary coil 110 enables a retinal prosthesis to be implemented which has no wiring or tethers from the intraocular device to the extraocular space. This arrangement allows the sclera 30 to completely heal after surgery, and return to its normal roles such as providing a barrier to intraocular infection and the like.

In some applications the interface between the retinal tack 160 and the flexible wiring 140 may suffer from long term reliability problems. These might be addressed by the implantation of a pre-connected and fully sealed connection between the two; rather than the use of retinal tacks.

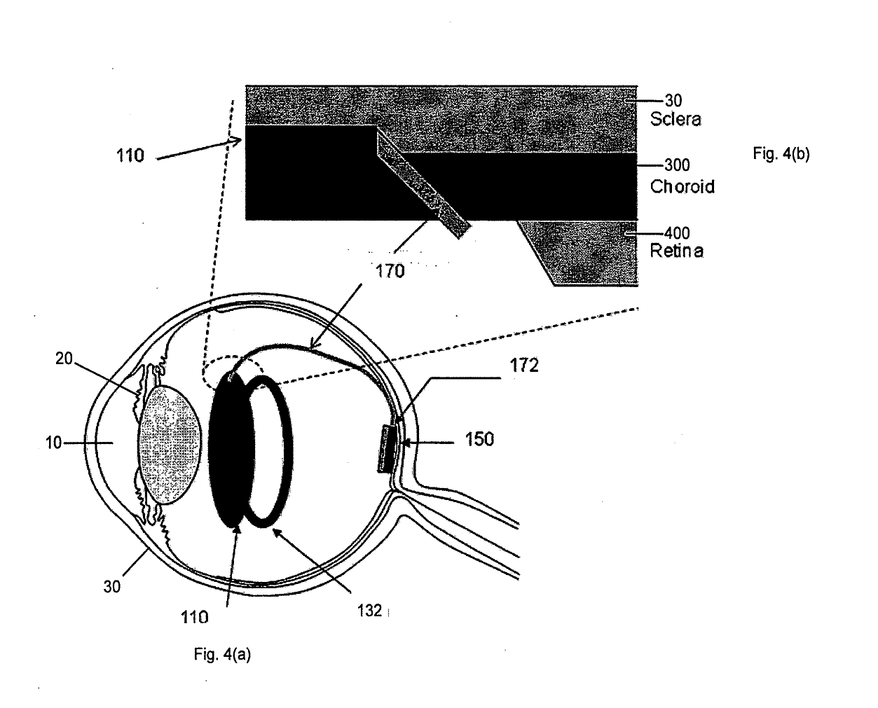

This might be achieved by use of a trans-vitreous link as shown in FIGS. 4(a) and 4(b). In this variation flexible wiring 170 extends from the secondary coil 110 through the vitreous humor directly to a connection 172 on the exposed surface of the implanted device 150. This allows the electrical connection 172 between the flexible link 170 and the implant 150 to be fabricated in an integrated manner before implantation, and therefore will be highly reliable.

Another alternative involves placement of an intermediate coil onto the outer surface of the sclera 30. This effectively eliminates any relative motions between the secondary coil 110 and the intermediate coil 132 due to eye movements. This increases the efficiency of the inductive link since wireless power transfer is dependent on the amount of overlap between the coils.

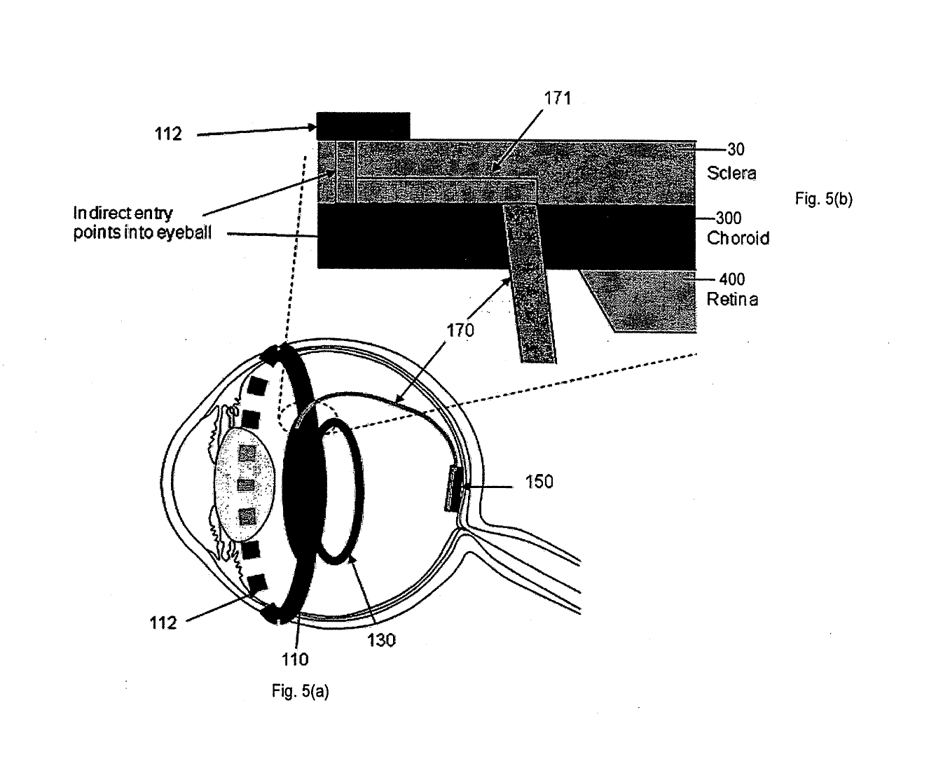

In the event that it is necessary for a transcleral link to be made, it is beneficial to run the link through the sclera 30, along between the sclera and the choroid 300 and then through the choroid, rather than penetrating directly through both layers of the eyeball. The link could run under the sclera 30 for up to half the perimeter of the eyeball before penetrating the choroid 300. This separation of the puncture through the sclera 30 and choroid 300 reduces the possibility of inner ocular infection and assists post-surgical recovery. For instance the secondary coil 110 could be mounted on a sclera buckle 112 and the assembly be placed epi-sclerally, as shown in FIG. 5. The benefit of placing the secondary coil 110 epi-sclerally is to enable a larger diameter coil to be realized, thus improving power transfer efficiency between the primary and secondary coils. Depending on the size of the secondary coil, this modification can potentially replace the need for an intermediate coil.

In order to implant the devices described with reference to FIGS. 4 and 5, the following surgical procedure maybe performed:

A sclera flap 500 is cut above the lateral eye muscle and the flab folded backward to expose the choroid 300 below.

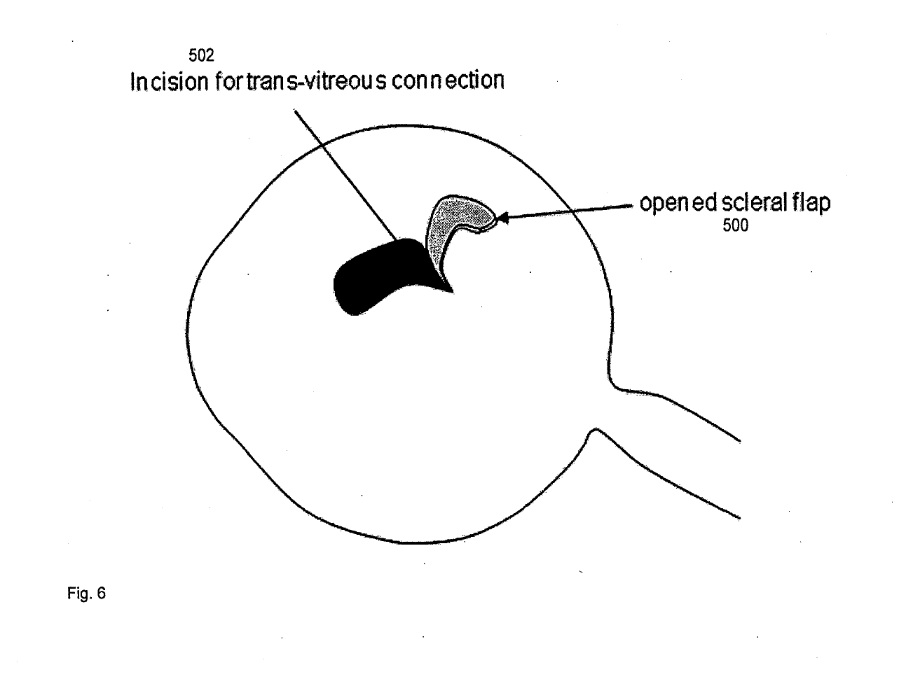

An incision 502 on the choroid 300 is then made, as shown in FIG. 6. The location of this incision should be made close to the pars plana region away from the retina 400 layer to avoid damage to the retina.

The implant 150 which has been integrated into the flexible trans-vitreous link 170 beforehand is then inserted through the incision.

Once the insertion is complete, the sclera flap 500 is closed. In the case of FIG. 4(a), closure of flap 500 is achieved by it being sutured back to the surrounding sclera 30 and fully sealed; as shown in FIG. 4(b). For the arrangement of FIG. 5(a), it is necessary to leave part of the flap open to allow for a trans-scleral link 171 to be connected to the secondary coil 110 which is mounted on the scleral buckle 112.

Another alternative arrangement involves the use of a single intermediate coil 180 placed between the skin and the zygomatic bone, in the vicinity of a primary 120 and a secondary 110; see FIG. 7.

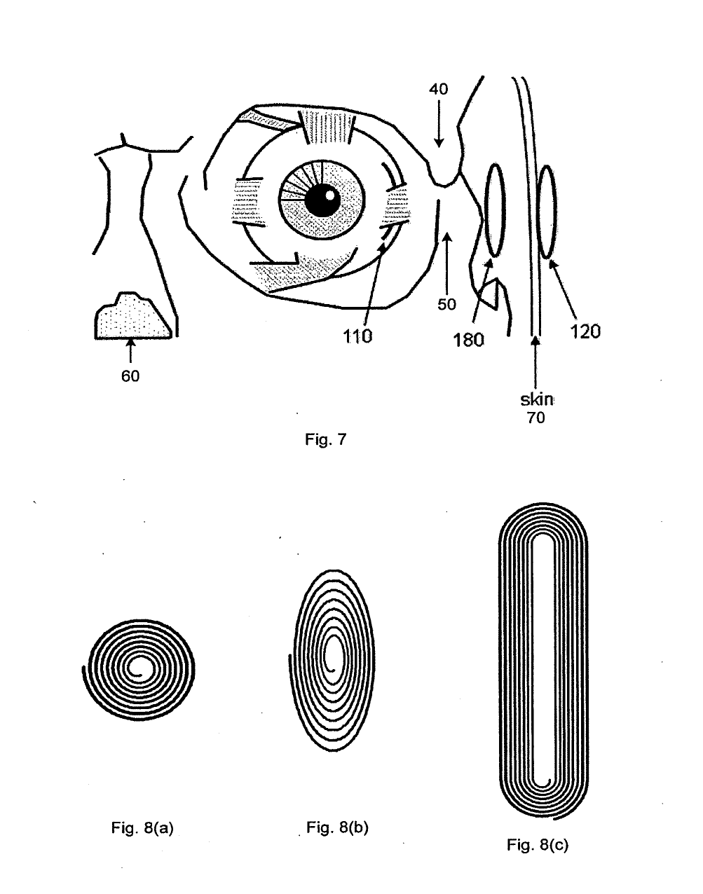

Many different configurations are possible for the power transfer coils 110, 120, 130, 132, 180. Generally, a coil consists of a continuous conductive track which spirals outwards resulting in a number of turns. The shape of the coil may be circular, oval or oblong as depicted in FIGS. 8(a), (b) and (c) respectively. The amount of required power transfer and ease of surgical procedure are factors that are taken into account when determining the size and shape of the coils.

It will be appreciated that a sympathetic combination of coil configuration and location, surgical and patient friendly design, and reliable mechanical and electrical connections are required to implement feasible inductive power transfer to a retinal implant.

It will also be appreciated by those skilled in the art that numerous variations and modifications may be made to the invention as described above without departing from the spirit or scope of the invention as broadly described. The present embodiments are, therefore, to be considered in all respects as illustrative and not restrictive.

What is claimed is:

1. A method for implanting a retinal prosthesis, comprising:
implanting a retinal electrode array in an eye;
implanting a receiving coil sub-sclerally, wherein the receiving coil is flexible and able to conform to curvature of a sclera of the eye when implanted; and
electrically connecting the retinal electrode array to the receiving coil,
such that power or data signals, or both, received by the receiving coil from a remote transmitting coil are automatically provided to the retinal electrode array.

2. The method according to claim 1, wherein implanting the retinal electrode array in the eye comprises implanting the retinal electrode array epi-retinally near a fovea in a macular region of a retina of the eye.

3. The method according to claim 1, wherein the method further comprises implanting one or more retinal self-locking tacks to mechanically secure the retinal electrode array in an epi-retinal position, and to complete an electrical connection between the receiving coil and the electrode array.

4. The method according to claim 3, wherein the one or more retinal self-locking tacks are formed of an insulating material, such as a ceramic, and house a conductor that spans electrical contacts at each end of the one or more retinal self-locking tacks.

5. The method according to claim 3, wherein the method further comprises arranging the one or more retinal self-locking tacks to conduct power or data signals, or both, from the receiving coil through a choroid or a retina, or both, of the eye to the electrode array.

6. The method according to claim 4, wherein the conductor carries data signals in both directions, to and from the retinal electrode array.

7. The method according to claim 1, wherein implanting the retinal electrode array comprises implanting the retinal electrode array in a suprachoroidal space of the eye.

8. The method according to claim 1, wherein the receiving coil is intrinsically flexible.

9. The method according to claim 1, wherein the method comprises the step of forming or mounting the receiving coil on a flexible substrate.

10. The method according to claim 1, wherein implanting the receiving coil comprises locating the receiving coil between a sclera and a choroid of the eye.

11. The method according to claim 10, wherein implanting the receiving coil comprises locating the receiving coil supra-choroidally and epi-sclerally.

12. The method according to claim 1, wherein electrically connecting the receiving coil to the retinal electrode array is by the use of flexible wiring.

13. The method according to claim 12, wherein the method further comprises implanting one or more retinal self-locking tacks to mechanically secure the retinal electrode array in an epi-retinal position, wherein one or more of the self-locking tacks complete an electrical connection between the receiving coil and the retinal electrode array, and implanting the flexible wiring sub-sclerally between the receiving coil and the one or more retinal self-locking tacks.

14. The method according to claim 12, wherein the flexible wiring is adapted for implanting sub-sclerally between the receiving coil and the retinal electrode array.

15. The method according to claim 12, wherein the flexible wiring from the receiving coil intrudes through a choroid and a vitreous humor to the retinal electrode array.

16. The method according to claim 15, wherein the flexible wiring does not pass through any part of a retina of the eye.

17. The method according to claim 1, wherein the remote transmitting coil is adapted to transmit power and data signals by inductive coupling to the receiving coil.

18. The method according to claim 17, wherein the method further comprises locating the remote transmitting coil externally to a body of the person having the eye.

19. The method according to claim 17, wherein the method further comprises implanting one or more intermediate coils to relay power or data signals, or both, from the remote transmitting coil to the receiving coil.

20. The method according to claim 19, wherein implanting the one or more intermediate coils comprises locating a first intermediate coil on an outer surface of the sclera of the eye.

21. The method according to claim 19, wherein the one or more intermediate coils comprises a first intermediate coil and a second intermediate coil, wherein the method comprises:
implanting the first intermediate coil in a zygomatic bone for receiving power from the remote transmitting coil, and
implanting the second intermediate coil in an orbit of an ocular region for inductively relaying power from the first intermediate coil,
wherein the first and second intermediate coils are in wired connection.

22. An ocular implant, comprising:
a receiving coil for implanting sub-sclerally to inductively receive power or data signals, or both;
a retinal electrode array, and
wiring interconnecting the receiving coil and the electrode array,
wherein the receiving coil and the wiring are flexible and at least the receiving coil is able to conform to curvature of the sclera, when implanted in an eye.

23. A method for implanting a transcleral link wherein the transcleral link passes through a sclera of an eye and is run along between the sclera and a choroid of the eye before penetrating a choroid of the eye.
